(19) United States
(12) Patent Application Publication
(10) Pub. No.: US 2008/0312537 A1
(43) Pub. Date: Dec. 18, 2008

(54) COMPOSITE PIEZOELECTRIC MATERIAL, ULTRASONIC PROBE, ULTRASONIC ENDOSCOPE, AND ULTRASONIC DIAGNOSTIC APPARATUS

(75) Inventor: Hiroaki HYUGA, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/138,014

(22) Filed: Jun. 12, 2008

(30) Foreign Application Priority Data
Jun. 12, 2007 (JP) 2007-154704

(51) Int. Cl. A61B 8/00

(52) U.S. Cl. 600/459

(57) ABSTRACT

A composite piezoelectric material capable of reducing a peak temperature of a vibrator array to be used for transmitting or receiving ultrasonic waves in ultrasonic imaging. The composite piezoelectric material includes: plural piezoelectric materials arranged along a flat surface or curved surface; and an anisotropic heat conducting material having a higher coefficient of thermal conductivity in at least one direction and provided between the plural piezoelectric materials and/or at outer peripheries of the plural piezoelectric materials.

### BACKGROUND OF THE INVENTION

1. Field of the Invention

The present invention relates to a composite piezoelectric material to be used in an ultrasonic transducer array for transmitting or receiving ultrasonic waves.

Further, the present invention relates to an ultrasonic probe including such an ultrasonic transducer array and to be used when intracavitary scan or extracavitary scan is performed on an object to be inspected, and an ultrasonic endoscope to be used by being inserted into a body cavity of the object. Furthermore, the present invention relates to an ultrasonic diagnostic apparatus including such an ultrasonic probe or ultrasonic endoscope and a main body apparatus.

2. Description of a Related Art

In medical fields, various imaging technologies have been developed in order to observe the interior of an object to be inspected and make diagnoses. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, and so on.

The ultrasonic imaging is an image generation technology utilizing the nature of ultrasonic waves that the ultrasonic waves are reflected at a boundary between regions having different acoustic impedances (e.g., a boundary between structures). Typically, an ultrasonic diagnostic apparatus (or referred to as an ultrasonic imaging apparatus or an ultrasonic observation apparatus) is provided with an ultrasonic probe to be used in contact with the object or ultrasonic probe to be used by being inserted into a body cavity of the object. Alternatively, an ultrasonic endoscope in combination of an endoscope for optically observing the interior of the object and an ultrasonic probe for intracavity is also used.

Using such an ultrasonic probe or ultrasonic endoscope, an ultrasonic beam is transmitted toward the object such as a human body and ultrasonic echoes generated by the object are received, and thereby, ultrasonic image information is acquired. On the basis of the ultrasonic image information, ultrasonic images of structures (e.g., internal organs, diseased tissues, or the like) existing within the object are displayed on a display unit of the ultrasonic diagnostic apparatus.

In the ultrasonic probe, a vibrator (piezoelectric vibrator) having electrodes formed on both sides of a material that expresses piezoelectric effect (a piezoelectric material) is generally used as an ultrasonic transducer for transmitting or receiving ultrasonic waves. As the piezoelectric material, a piezoelectric ceramics represented by PZT (Pb(lead) zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam can be formed and transmitted in a desired direction. Further, the vibrator expands and contracts by receiving the propagating ultrasonic waves, and generates an electric signal. The electric signal is used as a reception signal of ultrasonic waves.

When ultrasonic waves are transmitted, drive signals having great energy are supplied to the ultrasonic transducers. Not the whole energy of the drive signals is converted into acoustic energy, and the considerable amount of energy turns into heat. Thus, there has been a problem of temperature rise of the ultrasonic probe during its use. However, the ultrasonic probe for medical use is used in direct contact with a living body of human or the like, and the surface temperature of the ultrasonic probe is requested to be 50° C. or less when the ultrasonic probe is left in the air at 23° C. and requested to be 43° C. or less in contact with the human body for safety reasons for preventing low-temperature burn or the like.

As a related technology, Japanese Patent Application Publication JP-A-5-244690 (Document 1) discloses an ultrasonic probe in which the temperature rise on the vibrator surface is suppressed. The ultrasonic probe comprises a probe main body including a piezoelectric vibrator having electrodes on both principal surfaces thereof and generating ultrasonic waves, an acoustic matching layer formed on one principal surface side of the piezoelectric vibrator, a backing material attached to the other principal surface side of the piezoelectric vibrator, a heat radiating base made of metal and holding the backing material, and a heat conducting thin film for connecting the heat radiating base and the electrode on the one principal surface of the piezoelectric vibrator, and is characterized in that a heat conducting material is connected to the heat radiating base such that the heat conducting material is led out to the outside of a case in which the probe main body is accommodated.

Japanese Patent Application Publication JP-P2007-7262A (Document 2) discloses a convex-type ultrasonic probe capable of sufficiently attenuating ultrasonic waves transmitted in a backing member having a convex curved surface from piezoelectric elements of plural channels toward the backside, having good heat radiation performance, and capable of relaxing the concentration of heat generation. The ultrasonic probe includes (i) plural channels arranged with desirable spaces in between and having piezoelectric elements and an acoustic matching layer formed on the piezoelectric elements, (ii) a backing material including a support having a convex curved surface and a coefficient of thermal conductivity of 70 W/(m·K) or more, and an acoustic absorbing layer adhered to the convex curved surface of the support and having a sheet-like shape with a homogeneous entire thickness on which the piezoelectric elements of the respective channels are mounted and grooves are formed in locations corresponding to the spaces of the channels, and (iii) an acoustic lens formed on the acoustic matching layers of the respective channels, and is characterized in that the relation t1/t2=6 to 20 is satisfied where the thickness of the acoustic absorbing layer is represented by t1 and the thickness of the piezoelectric element is represented by t2.

Japanese Patent Publication JP-B-3420954 (Japanese Patent Application Publication JP-P2000-184497A: Document 3) discloses an ultrasonic probe capable of efficiently radiating heat generated in a piezoelectric element. The ultrasonic probe includes piezoelectric elements, a backside load material provided on the backside of the piezoelectric elements, a heat conducting material provided between the piezoelectric element and the backside load material and having a higher coefficient of thermal conductivity than that of the backside load material, and a heat radiating material provided around the backside load material, and is characterized in that the heat conducting material and the heat radiating material are thermally connected.

Japanese Patent Application Publication JP-A-10-75953 (Document 4) discloses an ultrasonic probe that reduces the amount of heat transferred from an internal heat generator to a surface of the body. The ultrasonic probe includes piezoelectric vibrators and a single-layered or multilayered acoustic matching layer covering the piezoelectric vibrators, and is characterized in that at least one layer of the acoustic matching layer is made as an acoustic matching layer with low heat conductivity.

In this regard, the three main factors of temperature rise in transmission of ultrasonic waves using an ultrasonic probe are as follows.

(1) The vibration energy of a vibrator itself, which is supplied with a drive signal to expand and contract, is converted into heat within the vibrator (self-heating).
(2) The ultrasonic waves generated by the vibrator are absorbed by a backing material and converted into heat.
(3) The ultrasonic waves generated by the vibrator are multiply-reflected on the interface of an acoustic matching layer or acoustic lens, and finally converted into heat.

The most important factor of them is the factor (1). However, in Documents 1-3, the radiation efficiency is poor because the heat generated in the vibrator is released only through the interface between the vibrator and the backing material. That is, the piezoelectric ceramics such as PZT forming the vibrator is poor in heat conductivity, and the epoxy resin, silicone resin, urethane resin, or the like filling between plural vibrators are also poor in heat conductivity, and therefore, sufficient radiation is not expected. Accordingly, there has been a problem that radiation at the central part of the vibrator array becomes especially insufficient and causes a temperature distribution in which the temperature at the central part is higher than that of the other part, and the peak temperature becomes higher. Further, in Document 4, at least one acoustic matching layer is made as an acoustic matching layer with low heat conductivity, however, the temperature rise of the vibrator cannot be avoided unless the heat generated in the vibrator is efficiently transferred to the outside.

### SUMMARY OF THE INVENTION

The present invention has been achieved in view of the above-mentioned problems. A purpose of the present invention is to provide a composite piezoelectric material capable of reducing a peak temperature of a vibrator array to be used for transmitting or receiving ultrasonic waves in ultrasonic imaging. Further, the present invention provides an ultrasonic probe, ultrasonic endoscope, and ultrasonic diagnostic apparatus using such a composite piezoelectric material.

In order to accomplish the purpose, a composite piezoelectric material according to one aspect of the present invention comprises: plural piezoelectric materials arranged along a flat surface or curved surface; and an anisotropic heat conducting material having a higher coefficient of thermal conductivity in at least one direction and provided between the plural piezoelectric materials and/or at outer peripheries of the plural piezoelectric materials.

Further, an ultrasonic probe according to one aspect of the present invention is an ultrasonic probe to be used for transmitting or receiving ultrasonic waves, and comprises: a vibrator array including the composite piezoelectric material according to the present invention; an acoustic matching layer and/or an acoustic lens provided on a first surface of the vibrator array; and a backing material provided on a second surface opposite to the first surface of the vibrator array.

Furthermore, an ultrasonic endoscope according to one aspect of the present invention is an ultrasonic endoscope including an insertion part formed of a material having flexibility to be used by being inserted into a body cavity of an object to be inspected, and the ultrasonic endoscope comprises in the insertion part: a vibrator array including the composite piezoelectric material according to the present invention; an acoustic matching layer and/or an acoustic lens provided on a first surface of the vibrator array; a backing material provided on a second surface opposite to the first surface of the vibrator array; illuminating means for illuminating an interior of the body cavity of the object; and imaging means for optically imaging the interior of the body cavity of the object.

In addition, an ultrasonic diagnostic apparatus according to one aspect of the present invention comprises: the ultrasonic probe or ultrasonic endoscope according to the present invention; drive signal supply means for supplying drive signals to the vibrator array; and signal processing means for generating image data representing an ultrasonic image by processing reception signals outputted from the vibrator array.

According to the present invention, since the anisotropic heat conducting material having a higher coefficient of thermal conductivity in at least one direction is provided between the plural piezoelectric materials, the heat generated in the piezoelectric vibrator can be rapidly transferred to the outside in the at least one direction. Therefore, the peak temperature of the vibrator array to be used for transmitting or receiving ultrasonic waves in ultrasonic imaging can be reduced.

### BRIEF DESCRIPTION OF THE DRAWINGS

FIG. 1 is a perspective view schematically showing an internal structure of an ultrasonic probe according to one embodiment of the present invention;

FIG. 2 is a sectional view of the internal structure of the ultrasonic probe shown in FIG. 1 along a plane in parallel with the YZ-plane;

FIG. 3A is a plan view of a composite piezoelectric material in the ultrasonic probe according to the first embodiment of the present invention, and FIG. 3B is a perspective view of the composite piezoelectric material in the ultrasonic probe according to the first embodiment of the present invention;

FIG. 4 shows measurement results of surface temperature of the ultrasonic probe according to the first embodiment of the present invention in comparison with those in a conventional case;

FIG. 5 shows structures of piezoelectric vibrator in comparison between the first embodiment of the present invention and a modified example thereof;

FIG. 6 shows measurement results of surface temperature of the ultrasonic probe according to the modified example of the first embodiment of the present invention in comparison with those in a conventional case;

FIG. 7A is a plan view of a composite piezoelectric material in an ultrasonic probe according to the second embodiment of the present invention, and FIG. 7B is a perspective view of the composite piezoelectric material in the ultrasonic probe according to the second embodiment of the present invention;

FIG. 8A is a plan view of a composite piezoelectric material in an ultrasonic probe according to the third embodiment of the present invention, and FIG. 8B is a side view of the composite piezoelectric material in the ultrasonic probe according to the third embodiment of the present invention;

FIG. 9 is a plan view of a composite piezoelectric material in an ultrasonic probe according to the third embodiment of the present invention;

FIG. 10 shows measurement results of surface temperature of the ultrasonic probe according to the modified example of the third embodiment of the present invention in comparison with those in a conventional case;

FIG. 11A is a plan view of a composite piezoelectric material in an ultrasonic probe according to the fourth embodiment of the present invention, and FIG. 11B is a perspective view of the composite piezoelectric material in the ultrasonic probe according to the fourth embodiment of the present invention;

FIG. 12 shows materials and so on of the respective parts in the fourth embodiment of the present invention;

FIG. 13 shows measurement results of surface temperature of the ultrasonic probe according to the fourth embodiment of the present invention in comparison with those in a conventional case;

FIG. 14 shows materials and so on of the respective parts in the fifth embodiment of the present invention;

FIG. 15 shows measurement results of surface temperature of the ultrasonic probe according to the fifth embodiment of the present invention in comparison with those in a conventional case;

FIG. 16 shows measurement results of surface temperature of the ultrasonic probe according to a modified example of the fifth embodiment of the present invention in comparison with those in a conventional case;

FIG. 17A is a plan view of a composite piezoelectric material in an ultrasonic probe according to the sixth embodiment of the present invention, and FIG. 17B is a side view of the composite piezoelectric material in the ultrasonic probe according to the sixth embodiment of the present invention;

FIG. 18 shows measurement results of surface temperature of the ultrasonic probe according to the modified example of the sixth embodiment of the present invention in comparison with those in a conventional case;

FIG. 19 is a schematic diagram showing an appearance of an ultrasonic endoscope according to one embodiment of the present invention;

FIG. 20 is an enlarged schematic diagram showing the leading end of an insertion part shown in FIG. 19; and

FIG. 21 shows an ultrasonic diagnostic apparatus including the ultrasonic probe or the ultrasonic endoscope according to the respective embodiments of the present invention and an ultrasonic diagnostic apparatus main body.

### DESCRIPTION OF THE PREFERRED EMBODIMENTS

Hereinafter, preferred embodiments of the present invention will be explained in detail with reference to the drawings. The same reference numerals will be assigned to the same component elements and the description thereof will be omitted.

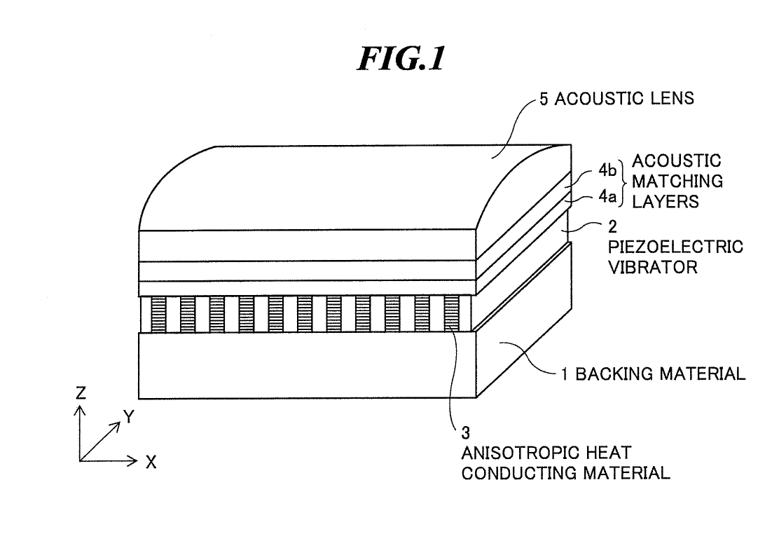

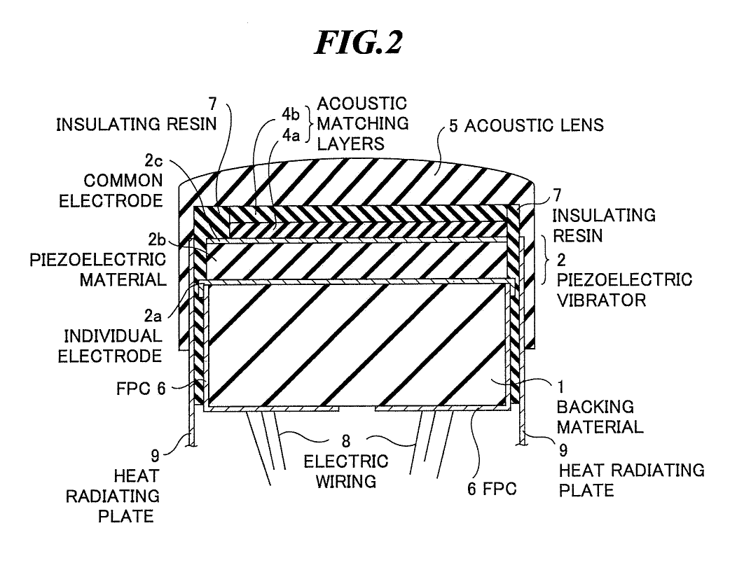

FIG. 1 is a perspective view schematically showing an internal structure of an ultrasonic probe according to the first embodiment of the present invention, and FIG. 2 is a sectional view of the internal structure of the ultrasonic probe shown in FIG. 1 along a plane in parallel with the YZ-plane. The ultrasonic probe is used when extracavitary scan is performed in contact with an object to be inspected or when intracavitary scan is performed by being inserted into a body cavity of the object.

As shown in FIGS. 1 and 2, the ultrasonic probe has a backing material 1, plural ultrasonic transducers (piezoelectric vibrators) 2 arranged on the backing material 1, an anisotropic heat conducting material 3 provided between those piezoelectric vibrators 2, one or plural acoustic matching layers (two acoustic matching layers 4a and 4b are shown in FIGS. 1 and 2) provided on the piezoelectric vibrators 2, an acoustic lens 5 provided on the acoustic matching layers according to need, two flexible printed circuit boards (FPCs) 6 fixed onto both side surfaces and the bottom surface of the backing material 1, insulating resins 7 formed on the side surfaces of the backing material 1, the piezoelectric vibrators 2, and the acoustic matching layers 4a and 4b via the FPCs 6, and electric wiring 8 connected to the FPCs 6. In FIG. 1, the FPCs 6 to electric wiring 8 are omitted and the acoustic lens 5 is partially cut for showing the arrangement of the piezoelectric vibrators 2. In the embodiment, the plural piezoelectric vibrators 2 arranged in the X-axis direction form a one-dimensional vibrator array.

As shown in FIG. 2, the piezoelectric vibrator 2 includes an individual electrode 2a formed on the backing material 1, a piezoelectric material 2b of PZT (Pb(lead) zirconate titanate) or the like formed on the individual electrode 2a, and a common electrode 2c formed on the piezoelectric material 2b. Typically, the common electrode 2c is commonly connected to the ground potential (GND). The individual electrodes 2a of the piezoelectric vibrators 2 are connected to the electric wiring 8 via printed wiring formed on the two FPCs 6 fixed onto the both side surfaces and the bottom surface of the backing material 1. The width of the piezoelectric material 2b (in the X-axis direction) is 100 μm, the length (in the Y-axis direction) is 5000 μm, and the thickness (in the Z-axis direction) is 300 μm. The polarization direction of the piezoelectric material 2b is the Z-axis direction.

Here, the plural piezoelectric materials 2b arranged in the X-axis direction and the anisotropic heat conducting material 3 provided between those piezoelectric materials 2b form a composite piezoelectric material. Further, in the present embodiment and the other embodiments, the anisotropic heat conducting material 3 may be provided at the outer peripheries of the plural piezoelectric materials 2b. Furthermore, at least one heat radiating plate (two heat radiating plates 9 are shown in FIG. 2) may be provided on the side surfaces of the backing material 1 and the piezoelectric vibrators 2 via the FPCs 6 and the insulating resins 7. In this case, the heat radiating plate 9 may be connected to a shield layer of a conducting material provided in a cable for connecting the ultrasonic probe to the ultrasonic diagnostic apparatus main body. As a material of the heat radiating plate 9, a metal having a high coefficient of thermal conductivity such as copper (Cu) is used. Further, as the insulating resin 7, a resin having a high coefficient of thermal conductivity is desirably used. The heat generated at the central part of the piezoelectric vibrator 2 transfers toward the side surface (in the Y-axis direction) via the anisotropic heat conducting material 3 and transfers to the heat radiating plate 9 via the insulating resin 7.

The backing material 1 is formed of a material having great acoustic attenuation such as an epoxy resin including ferrite powder, metal powder, or PZT powder, or rubber including ferrite powder, and promotes attenuation of unwanted ultrasonic waves generated from the plural piezoelectric vibrators 2. In the case of a convex array probe, the backing material 1 having a shape convex upward is used.

The plural ultrasonic vibrators 2 generate ultrasonic waves based on the drive signals respectively supplied from the ultrasonic diagnostic apparatus main body. Further, the plural ultrasonic vibrators 2 receive ultrasonic echoes propagating from the object and generate plural electric signals, respectively. The electric signals are outputted to the ultrasonic diagnostic apparatus main body and processed as reception signals of the ultrasonic echoes.

The acoustic matching layers 4a and 4b formed on the front surface of the ultrasonic vibrators 2 are formed of Pyrex (registered trademark) glass or an epoxy resin including metal powder, which easily propagates ultrasonic waves, for example, and provides matching of acoustic impedances between the object as a living body and the ultrasonic vibrators 2. Thereby, the ultrasonic waves transmitted from the ultrasonic vibrators 2 efficiently propagate within the object.

The acoustic lens 5 is formed of silicone rubber, for example, and focuses an ultrasonic beam transmitted from the ultrasonic transducer array 12 and propagating through the acoustic matching layers 4a and 4b at a predetermined depth within the object.

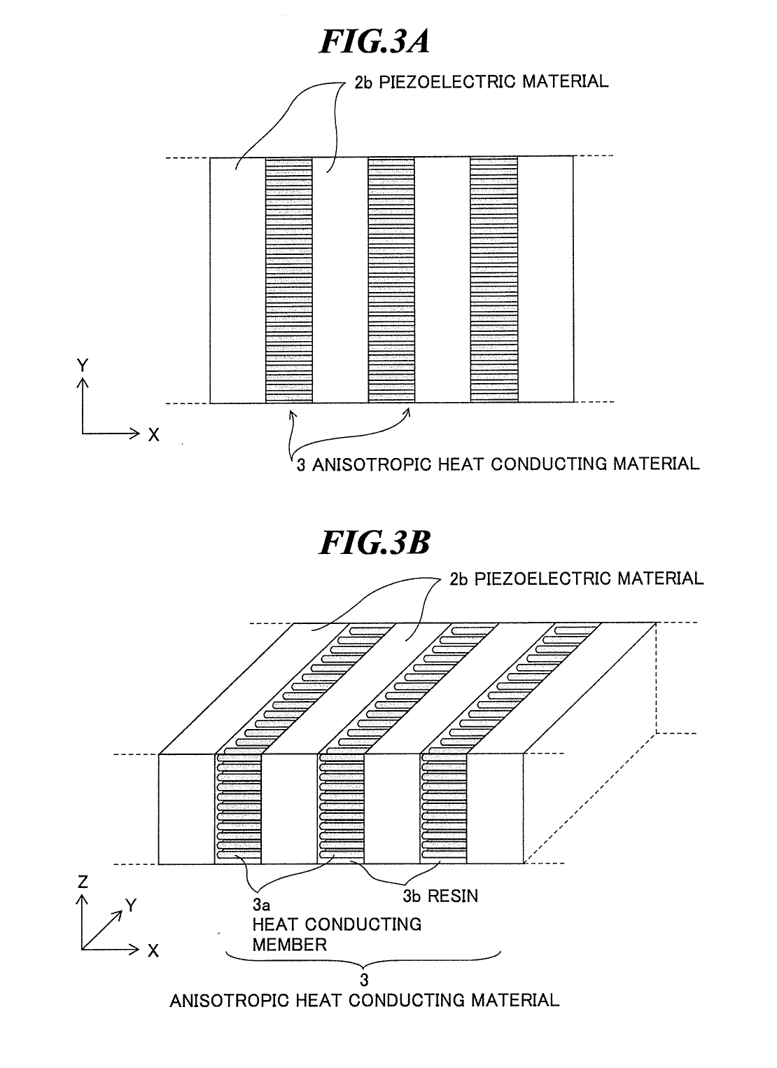

FIG. 3A is a plan view of the composite piezoelectric material in the ultrasonic probe according to the first embodiment of the present invention, and FIG. 3B is a perspective view of the composite piezoelectric material in the ultrasonic probe according to the first embodiment of the present invention. In the embodiment, in order to reduce the peak temperature by flattening the temperature distribution of the vibrator array, the anisotropic heat conducting material 3 is provided between the plural piezoelectric materials 2b forming the vibrator array as shown in FIG. 3A.

As shown in FIG. 3B, the anisotropic heat conducting material 3 includes plural heat conducting members 3a arranged such that the longitudinal direction thereof is substantially in parallel to the ultrasonic wave transmission and reception surface of the piezoelectric vibrator, and a resin 3b filling between the heat conducting members 3a. Although the resins 3b are generally not transparent, the heat conducting members 3a are shown through the resins 3b in FIGS. 3A, 3B and so on. The heat conducting member 3a may have a fibrous or rod-like shape for higher heat conductivity in one direction, or may have a planar shape for higher heat conductivity in two directions. The longitudinal direction of the heat conducting members 3a may not be necessary to be in parallel to the ultrasonic wave transmission and reception surface of the vibrator, but it is desirable that the angle formed by the heat conducting member 3a and the ultrasonic wave transmission and reception surface is 30° or less for flattening the temperature distribution of the vibrator array.

An inorganic material having a good coefficient of thermal conductivity is suitable for the chief material of the heat conducting member 3a, and a metal such as gold (Au), silver (Ag), copper (Cu), or aluminum (Al), or silicon carbide (SiC), aluminum nitride (AlN), tungsten carbide (WC), boron nitride (BN), alumina (aluminum oxide: Al₂O₃), carbon fiber, carbon nanotube, or the like may be used.

Among those materials, the materials except for the aluminum nitride or alumina as ceramics have conductivity, and a film of an insulating material is desirably formed on the surface thereof. The film may be formed by electrodeposition of an insulating resin on the surface of the chief material, application of an insulating resin thereto and curing it, or vapor-phase deposition by sputtering using an insulating material such as silicon oxide (SiO₂).

As the resin 3b, an epoxy resin, urethane resin, silicone resin, acrylic resin, or the like may be used. Further, in order to improve the coefficient of thermal conductivity, particles of diamond, black lead, metal, silicon carbide (SiC), aluminum nitride (AlN), tungsten carbide (WC), boron nitride (BN), or alumina (aluminum oxide: Al₂O₃) may be added to the resin 3b and mixed therewith.

As below, the case where a carbon fiber is used as the heat conducting member 3a and an epoxy resin is used as the resin 3b will be explained. As shown in FIGS. 3A and 3B, in the composite piezoelectric material used in the first embodiment of the present invention, the carbon fibers as the heat conducting members 3a are arranged substantially in parallel to the arrangement direction of the piezoelectric materials 2b (the X-axis direction). Thereby, the temperature distribution in the vibrator array is flattened. The longitudinal direction of the heat conducting members 3a may not be necessary to be in parallel to the X-axis direction, but it is desirable that the angle formed by the heat conducting member 3a and the X-axis direction is 30° or less for flattening the temperature distribution of the vibrator array.

A diameter of each carbon fiber is about 10 μm. The resins 3b are formed by pouring the epoxy resin into spaces between the plural fibers and curing it. The volume fraction of carbon fibers in the anisotropic heat conducting material 3 is preferably from 20% to 78%, and 50% in the embodiment.

The coefficient of thermal conductivity of carbon fiber is about 800 W/(m·K), and the coefficient of thermal conductivity of epoxy resin is about 0.2 W/(m·K). Therefore, the coefficient of thermal conductivity of the anisotropic heat conducting material 3 is about 400 W/(m·K) with respect to the longitudinal direction of the carbon fiber and about 0.4 W/(m·K) with respect to the direction perpendicular to the longitudinal direction of the carbon fiber, and they are greatly improved compared to about 0.2 W/(m·K) in the conventional case of the epoxy resin only. In the first embodiment, the coefficient of thermal conductivity is remarkably improved in the arrangement direction of the piezoelectric vibrators (the X-axis direction).

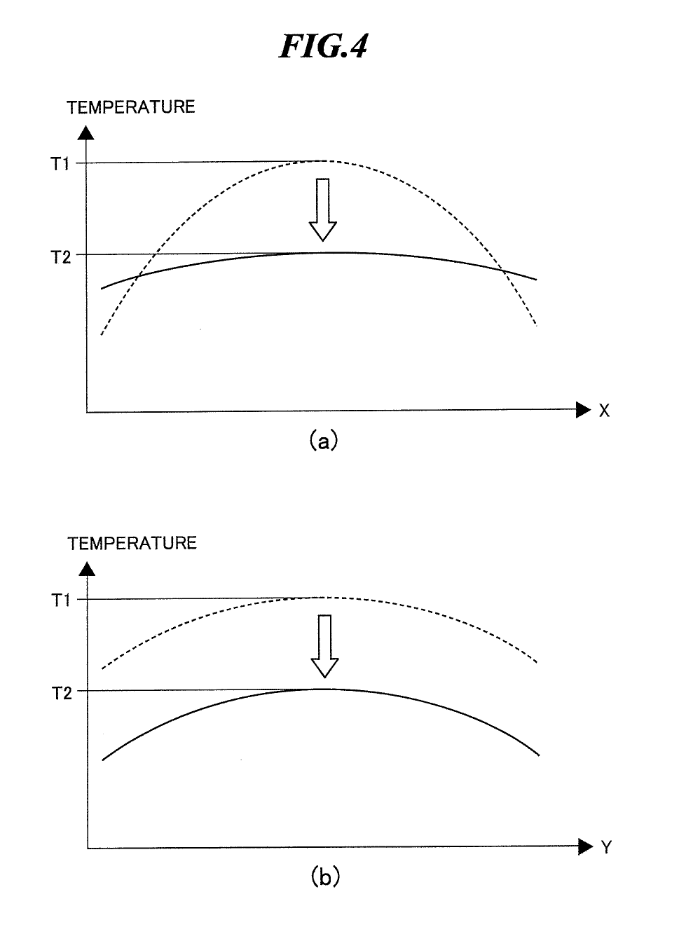

FIG. 4 shows measurement results of surface temperature of the ultrasonic probe according to the first embodiment of the present invention in comparison with those in a conventional case. The measurement was made by measuring the surface temperature of the acoustic lens in the air at a temperature of 23° C. In the ultrasonic probe used in surface temperature measurement, the heat radiating plate 9 shown in FIG. 2 is not provided. FIG. 4 (a) shows a temperature distribution in the X-axis direction, which passes through the point of peak temperature on the surface of the acoustic lens, and FIG. 4 (b) shows a temperature distribution in the Y-axis direction, which passes through the point of peak temperature on the surface of the acoustic lens.

In FIGS. 4 (a) and 4 (b), the broken lines show measurement results of surface temperature of the conventional ultrasonic probe, and solid lines show measurement results of surface temperature of the ultrasonic probe according to the first embodiment. The peak temperature T1 in the conventional ultrasonic probe was 39° C., while the peak temperature T2 in the ultrasonic probe according to the first embodiment was 30° C., and accordingly, it is known that the peak temperature is reduced by providing the anisotropic heat conducting material between the plural piezoelectric materials. Further, the surface temperature of the ultrasonic probe can be further reduced by providing the heat radiating plates 9 shown in FIG. 2.

Next, a modified example of the first embodiment of the present invention will be explained. In the modified example, the piezoelectric vibrator has a multilayered structure, and the rest of the configuration is the same as that in the first embodiment.

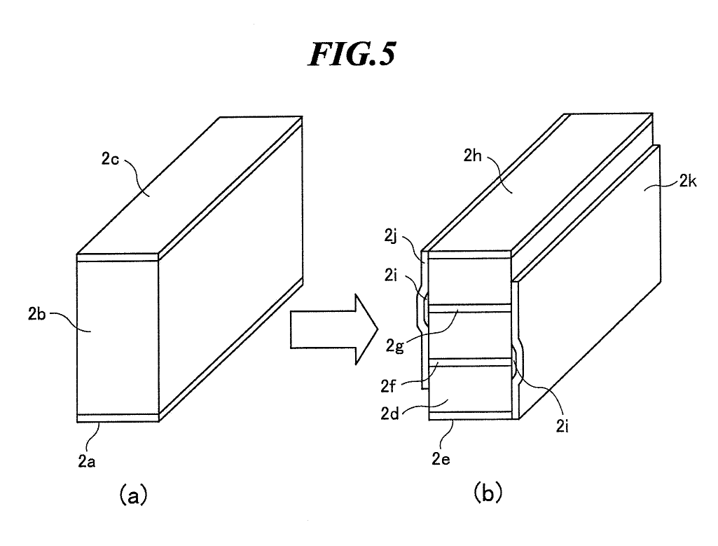

FIG. 5 shows structures of piezoelectric vibrator in comparison between the first embodiment of the present invention and the modified example thereof. In the first embodiment shown in FIG. 5 (a), a piezoelectric vibrator includes an individual electrode 2a, a piezoelectric material 2b formed on the individual electrode 2a, and a common electrode 2c formed on the piezoelectric material 2b.

On the other hand, in the modified example of the first embodiment shown in FIG. 5 (b), a piezoelectric vibrator includes plural piezoelectric material layers 2d formed of PZT or the like, a lower electrode layer 2e, internal electrode layers 2f and 2g alternately inserted between the plural piezoelectric material layers 2d, an upper electrode layer 2h, insulating films 2i, and side electrodes 2j and 2k.

Here, the lower electrode layer 2e is connected to the side electrode 2k at the right side in the drawing and insulated from the side electrode 2j at the left side in the drawing. The upper electrode layer 2h is connected to the side electrode 2j and insulated from the side electrode 2k. Further, the internal electrode layer 2f is connected to the side electrode 2j and insulated from the side electrode 2k by the insulating film 2i. On the other hand, the internal electrode layer 2g is connected to the side electrode 2k and insulated from the side electrode 2j by the insulating film 2i. The plural electrodes of an ultrasonic transducer are formed in this fashion, three pairs of electrodes for applying electric fields to the three layers of piezoelectric vibrator layers 2d are connected in parallel. The number of piezoelectric vibrator layers is not limited to three, but may be two or four or more.

In the multilayered piezoelectric vibrator, the area of opposed electrodes becomes larger than that of the single-layered element, and the electric impedance becomes lower. Therefore, the multilayered piezoelectric vibrator operates more efficiently for the applied voltage than the single-layered piezoelectric vibrator having the same size. Specifically, given that the number of piezoelectric material layers is N, the number of the multilayered piezoelectric vibrator is N-times the number of piezoelectric material layers of the single-layered piezoelectric vibrator, and the thickness of each layer of the multilayered piezoelectric vibrator is 1/N of the thickness of each layer of the single-layered piezoelectric vibrator, and therefore, the electric impedance of the multilayered piezoelectric vibrator is 1/N²-times the electric impedance of the single-layered piezoelectric vibrator. Accordingly, the electric impedance of piezoelectric vibrator can be adjusted by increasing or decreasing the number of stacked piezoelectric material layers, and thus, the electric impedance matching between a drive circuit or preamplifier and itself is easily provided and the sensitivity can be improved. On the other hand, the capacitance is increased due to the stacked form of the piezoelectric vibrator, the amount of heat generated in each piezoelectric vibrator becomes larger.

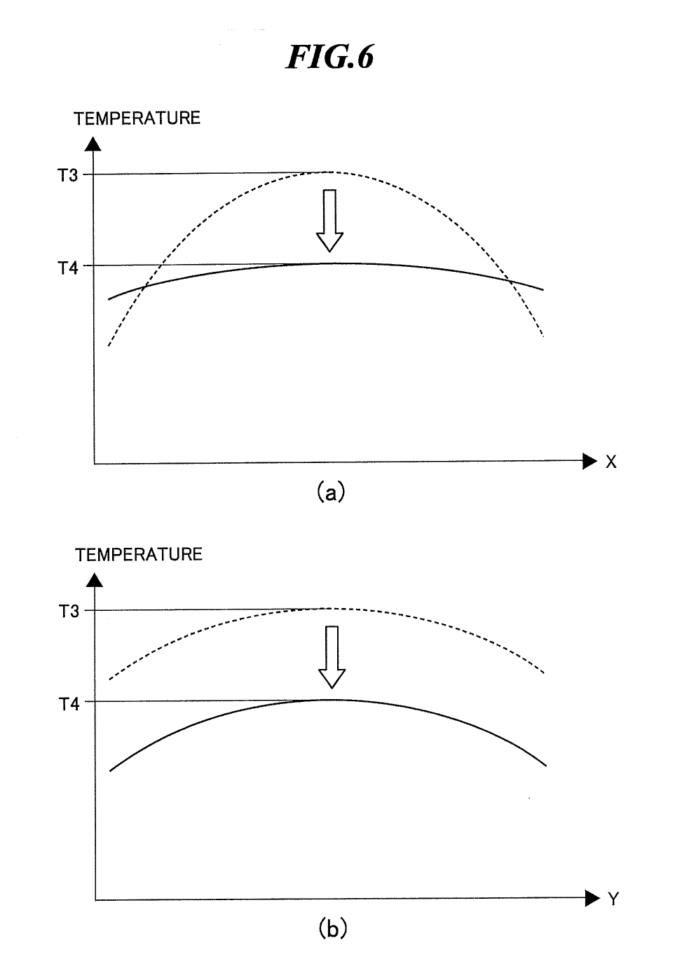

FIG. 6 shows measurement results of surface temperature of the ultrasonic probe according to the modified example of the first embodiment of the present invention in comparison with those in a conventional case. The measurement was made by measuring the surface temperature of the acoustic lens in the air at a temperature of 23° C. FIG. 6 (a) shows a temperature distribution in the X-axis direction, which passes through the point of peak temperature on the surface of the acoustic lens, and FIG. 6 (b) shows a temperature distribution in the Y-axis direction, which passes through the point of peak temperature on the surface of the acoustic lens.

In FIGS. 6 (a) and 6 (b), the broken lines show measurement results of surface temperature of the conventional ultrasonic probe, and solid lines show measurement results of surface temperature of the ultrasonic probe according to the modified example of the first embodiment. The peak temperature T3 in the conventional ultrasonic probe was 77° C., while the peak temperature T4 in the ultrasonic probe according to the modified example of the first embodiment was 46° C. According to the modified example of the first embodiment, even when the amounts of heat generated in multilayered piezoelectric vibrators become larger, the temperature distribution in the vibrator array can be flattened by providing the anisotropic heat conducting material between the plural piezoelectric materials, and thereby, the peak temperature rise in the vibrator array can be suppressed.

Next, the second embodiment of the present invention will be explained. In the second embodiment, the orientation of heat conducting members is different from that in the first embodiment, but the rest of the configuration is the same as that in the first embodiment.

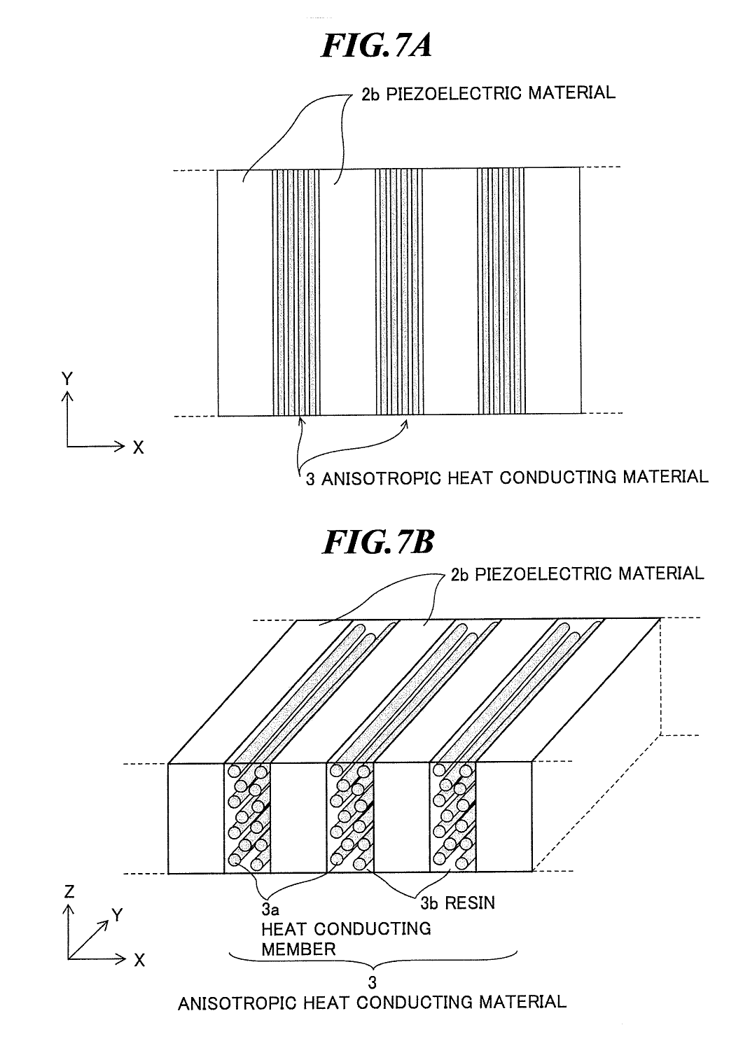

FIG. 7A is a plan view of a composite piezoelectric material in an ultrasonic probe according to the second embodiment of the present invention, and FIG. 7B is a perspective view of the composite piezoelectric material in the ultrasonic probe according to the second embodiment of the present invention.

As shown in FIGS. 7A and 7B, in the composite piezoelectric material used in the second embodiment of the present invention, the carbon fibers as the heat conducting members 3a are arranged substantially in parallel to the longitudinal direction of the piezoelectric materials 2b (the Y-axis direction). Thereby, the temperature distribution in the vibrator array is flattened. The longitudinal direction of the heat conducting members 3a may not be necessary to be in parallel to the Y-axis direction, but it is desirable that the angle formed by the heat conducting member 3a and the Y-axis direction is 30° or less for flattening the temperature distribution of the vibrator array.

The diameter of each carbon fiber is about 10 μm. The resins 3b are formed by pouring the epoxy resin into spaces between the plural fibers and curing it. The volume fraction of carbon fibers in the anisotropic heat conducting material 3 is preferably from 20% to 78%, and 50% in the embodiment. In the second embodiment, especially, the coefficient of thermal conductivity is remarkably improved in the arrangement direction of the piezoelectric vibrators (the Y-axis direction).

Next, the third embodiment of the present invention will be explained. In the third embodiment, plural piezoelectric vibrators arranged in the X-axis direction and the Y-axis direction form a two-dimensional vibrator array, and the acoustic lens 5 shown in FIGS. 1 and 2 is not formed.

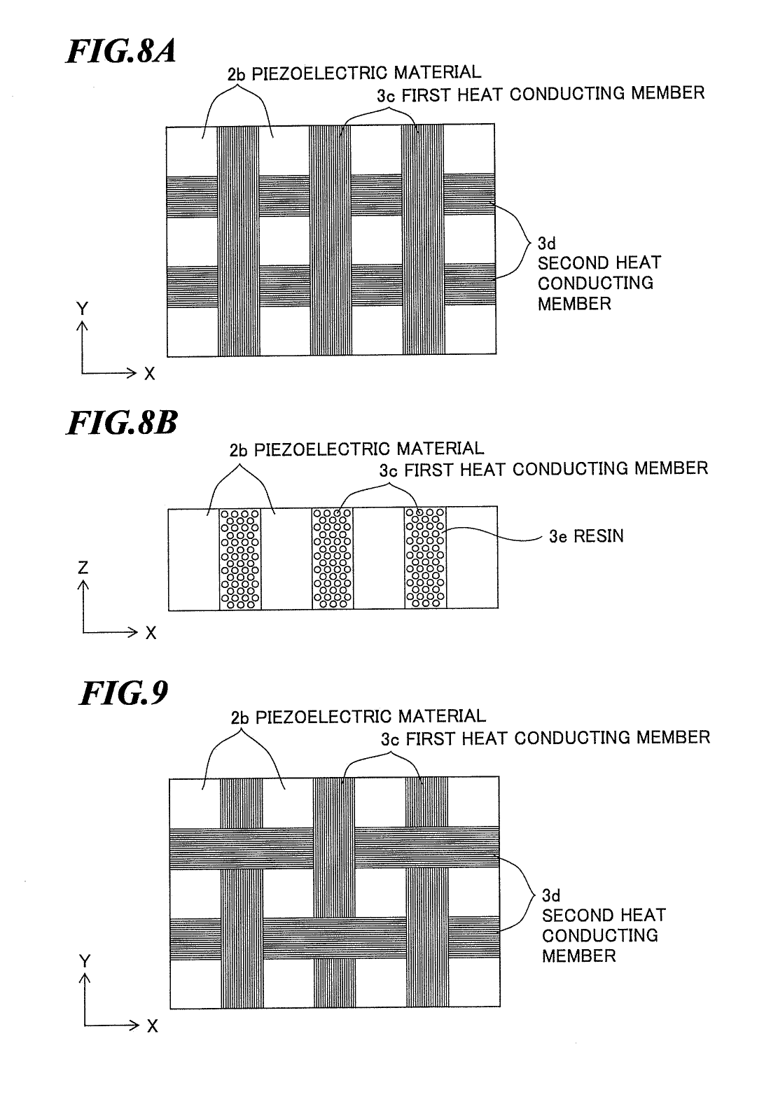

FIG. 8A is a plan view of a composite piezoelectric material in an ultrasonic probe according to the third embodiment of the present invention, and FIG. 8B is a side view of the composite piezoelectric material in the ultrasonic probe according to the third embodiment of the present invention. Here, sides of a piezoelectric material 2b (in the X-axis direction and the Y-axis direction) are 250 μm, and the thickness of the piezoelectric material 2b (in the Z-axis direction) is 600 μm. The polarization direction of the piezoelectric material 2b is the Z-axis direction.

Anisotropic heat conducting members provided between plural piezoelectric materials 2b forming a piezoelectric vibrator array includes plural first heat conducting members 3c arranged in plural rows such that the longitudinal direction is substantially in parallel to the Y-axis direction, plural second heat conducting members 3d arranged between the plural rows of the heat conducting members 3c such that the longitudinal direction is substantially in parallel to the X-axis direction, and resins 3e filling between the heat conducting members 3c and 3d. Each of the heat conducting members 3c and 3d has a fibrous or rod-like shape for higher heat conductivity in one direction. The longitudinal direction of the heat conducting members 3c and 3d may not be necessary to be in parallel to the Y-axis direction and the X-axis direction, but it is desirable that the angles formed by the heat conducting members 3c and 3d and the Y-axis direction and the X-axis direction are 300 or less, respectively, for flattening the temperature distribution of the vibrator array.

Further, as shown in FIG. 9, the first heat conducting members 3c and the second heat conducting members 3d may be alternately crossed. In this case, the length of the second heat conducting members 3d can be made relatively long. When such a structure is fabricated, the first heat conducting members 3c and the second heat conducting members 3d are inter knitted in advance, and then, they are inserted into spaces between the plural piezoelectric materials 2b.

The material of the heat conducting members 3c and 3d is the same as that of the heat conducting members 3a in the first embodiment, and the material of the resin 3e is the same as that of the resin 3b in the first embodiment. As below, the case where carbon fibers are used as the heat conducting members 3c and 3d and an epoxy resin is used as the resin 3e will be explained. The diameter of each carbon fiber is about 10 μm. The resins 3e are formed by pouring the epoxy resin into spaces between the plural fibers and curing it. The volume fraction of carbon fibers in the anisotropic heat conducting material 3 is preferably from 20% to 78%, and 40% in the embodiment.

The coefficient of thermal conductivity of carbon fiber is about 800 W/(m·K), and the coefficient of thermal conductivity of epoxy resin is about 0.2 W/(m·K). Therefore, the coefficient of thermal conductivity of the anisotropic heat conducting material 3 is about 320 W/(m·K) with respect to the longitudinal direction of the carbon fiber and about 0.33 W/(m·K) with respect to the direction perpendicular to the longitudinal direction of the carbon fiber, and they are greatly improved compared to about 0.2 W/(m·K) in the conventional case of the epoxy resin only. In the third embodiment, the coefficients of thermal conductivity are remarkably improved in both X-axis direction and Y-axis direction.

Next, a modified example of the third embodiment of the present invention will be explained. In the modified example, the piezoelectric vibrator has a multilayered structure as is in the case shown in FIG. 5, and the rest of the configuration is the same as that in the third embodiment.

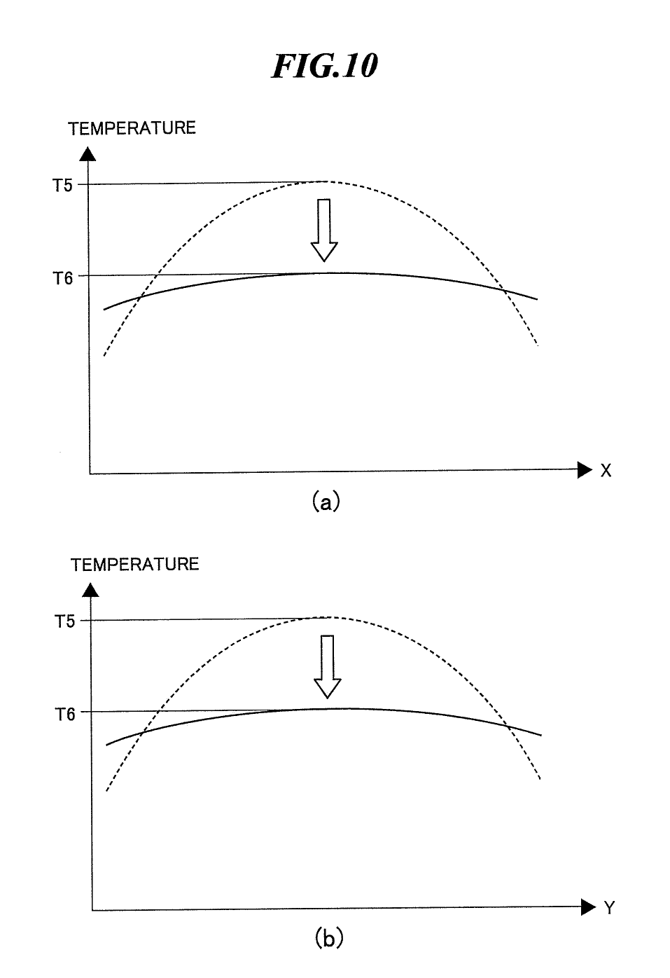

FIG. 10 shows measurement results of surface temperature of the ultrasonic probe according to the modified example of the third embodiment of the present invention in comparison with those in a conventional case. The measurement was made by measuring the surface temperature of the acoustic lens in the air at a temperature of 23° C. FIG. 10 (a) shows a temperature distribution in the X-axis direction, which passes through the point of peak temperature on the surface of the acoustic lens, and FIG. 10 (b) shows a temperature distribution in the Y-axis direction, which passes through the point of peak temperature on the surface of the acoustic lens.

In FIGS. 10 (a) and 10 (b), the broken lines show measurement results of surface temperature of the conventional ultrasonic probe, and solid lines show measurement results of surface temperature of the ultrasonic probe according to the modified example of the third embodiment. The peak temperature T5 in the conventional ultrasonic probe was 70° C., while the peak temperature T6 in the ultrasonic probe according to the modified example of the first embodiment was 42° C. According to the modified example of the third embodiment, even when the amounts of heat generated in multilayered piezoelectric vibrators become larger, the temperature distribution in the vibrator array can be flattened by providing the anisotropic heat conducting material between the plural piezoelectric materials, and thereby, the peak temperature rise in the vibrator array can be suppressed.

Next, the fourth embodiment of the present invention will be explained. In the fourth embodiment, the orientation of heat conducting members is different from that in the first embodiment, but the rest of the configuration is the same as that in the first embodiment.

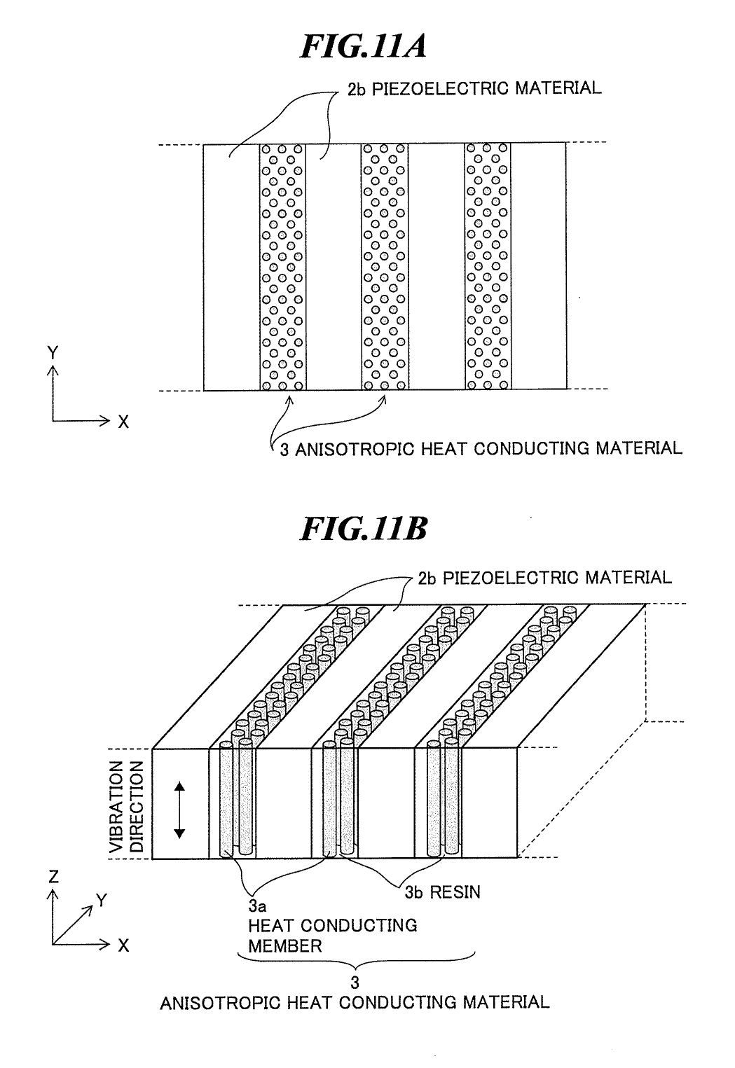

FIG. 11A is a plan view of a composite piezoelectric material in an ultrasonic probe according to the fourth embodiment of the present invention, and FIG. 11B is a perspective view of the composite piezoelectric material in the ultrasonic probe according to the fourth embodiment of the present invention. In the embodiment, in order to rapidly release the heat generated in the piezoelectric vibrator to the backing material 1 (FIGS. 1 and 2) to reduce the peak temperature, the anisotropic heat conducting material 3 is provided between the plural piezoelectric materials 2b forming the vibrator array as shown in FIG. 11A.

The anisotropic heat conducting material 3 includes plural heat conducting members 3a arranged such that the longitudinal direction is substantially in parallel to the vibration direction of the piezoelectric vibrator (the Z-axis direction), and resins 3b filling between the heat conducting members 3a. The heat conducting member 3a has a fibrous or rod-like shape for higher heat conductivity in one direction. The longitudinal direction of the heat conducting members 3a may not be necessary to be in parallel to the Z-axis direction, but it is desirable that the angle formed by the heat conducting member 3a and the Z-axis direction is 300 or less for releasing the heat generated in the piezoelectric vibrators to the backing material.

As below, the case where a carbon fiber is used as the heat conducting member 3a and an epoxy resin is used as the resin 3b will be explained. The diameter of each carbon fiber is about 10 μm. The resins 3b are formed by pouring the epoxy resin into spaces between the plural fibers and curing it. The volume fraction of carbon fibers in the anisotropic heat conducting material 3 is preferably from 20% to 78%, and 50% in the embodiment.

The coefficient of thermal conductivity of carbon fiber is about 800 W/(m·K), and the coefficient of thermal conductivity of epoxy resin is about 0.2 W/(m·K). Therefore, the coefficient of thermal conductivity of the anisotropic heat conducting material 3 is about 400 W/(m·K) with respect to the longitudinal direction of the carbon fiber and about 0.4 W/(m·K) with respect to the direction perpendicular to the longitudinal direction of the carbon fiber, and they are greatly improved compared to about 0.2 W/(m·K) in the conventional case of the epoxy resin only. In the fourth embodiment, the coefficient of thermal conductivity is remarkably improved in the vibration direction of the piezoelectric vibrators (the Z-axis direction).

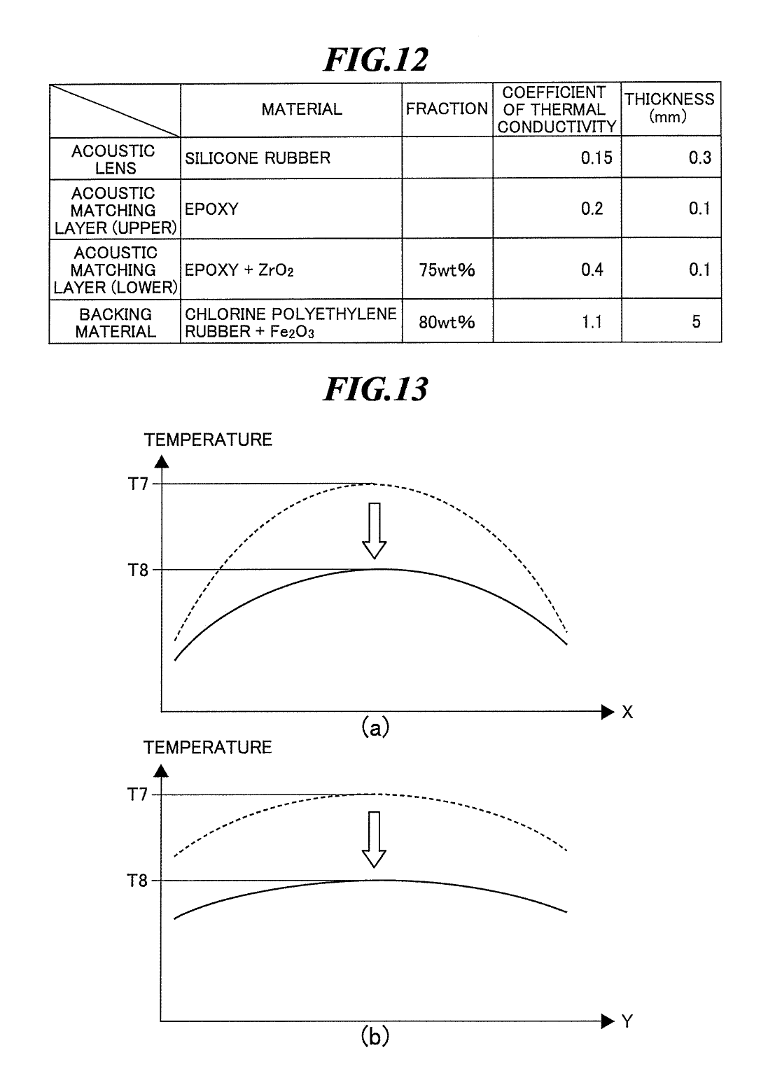

FIG. 12 shows materials and so on of the respective parts in the fourth embodiment of the present invention. As the backing material 1 shown in FIGS. 1 and 2, a material formed by mixing 80 wt % in weight fraction of ferric oxide (Fe₂O₃) in chlorine polyethylene rubber is used. The backing material 1 has a coefficient of thermal conductivity of about 1.1 W/(m·K), and a thickness of 5 mm. As the acoustic matching layer (the lower layer) 4a, a material formed by mixing 75 wt % in weight fraction of zirconia (ZrO₂) in epoxy resin is used. The acoustic matching layer (the lower layer) 4a has a coefficient of thermal conductivity of about 0.4 W/(m·K), and a thickness of 0.1 mm. As the acoustic matching layer (the upper layer) 4b, epoxy resin is used. The acoustic matching layer (the upper layer) 4b has a coefficient of thermal conductivity of about 0.2 W/(m·K), and a thickness of 0.1 mm. As the acoustic lens 5, silicone rubber is used. The acoustic lens 5 has a coefficient of thermal conductivity of about 0.15 W/(m·K), and a thickness of 0.3 mm.

FIG. 13 shows measurement results of surface temperature of the ultrasonic probe according to the fourth embodiment of the present invention in comparison with those in a conventional case. The measurement was made by measuring the surface temperature of the acoustic lens in the air at a temperature of 23° C. FIG. 13 (a) shows a temperature distribution in the X-axis direction, which passes through the point of peak temperature on the surface of the acoustic lens, and FIG. 13 (b) shows a temperature distribution in the Y-axis direction, which passes through the point of peak temperature on the surface of the acoustic lens.

In FIGS. 13 (a) and 13 (b), the broken lines show measurement results of surface temperature of the conventional ultrasonic probe, and solid lines show measurement results of surface temperature of the ultrasonic probe according to the fourth embodiment. The peak temperature T7 in the conventional ultrasonic probe was 39° C., while the peak temperature T8 in the ultrasonic probe according to the fourth embodiment was 34° C., and accordingly, it is known that the peak temperature is reduced by providing the anisotropic heat conducting material between the plural piezoelectric materials.

Next, the fifth embodiment of the present invention will be explained. In the fifth embodiment, as the backing material 1 shown in FIGS. 1 and 2, a material having a higher coefficient of thermal conductivity than those of the acoustic matching layers 4a and 4b and the acoustic lens 5 is used. It is preferable that the coefficient of thermal conductivity of the backing material 1 is not less than 10 times a coefficient of thermal conductivity of the acoustic matching layers 4a and 4b or the acoustic lens 5. The rest of the configuration is the same as that of the fourth embodiment.

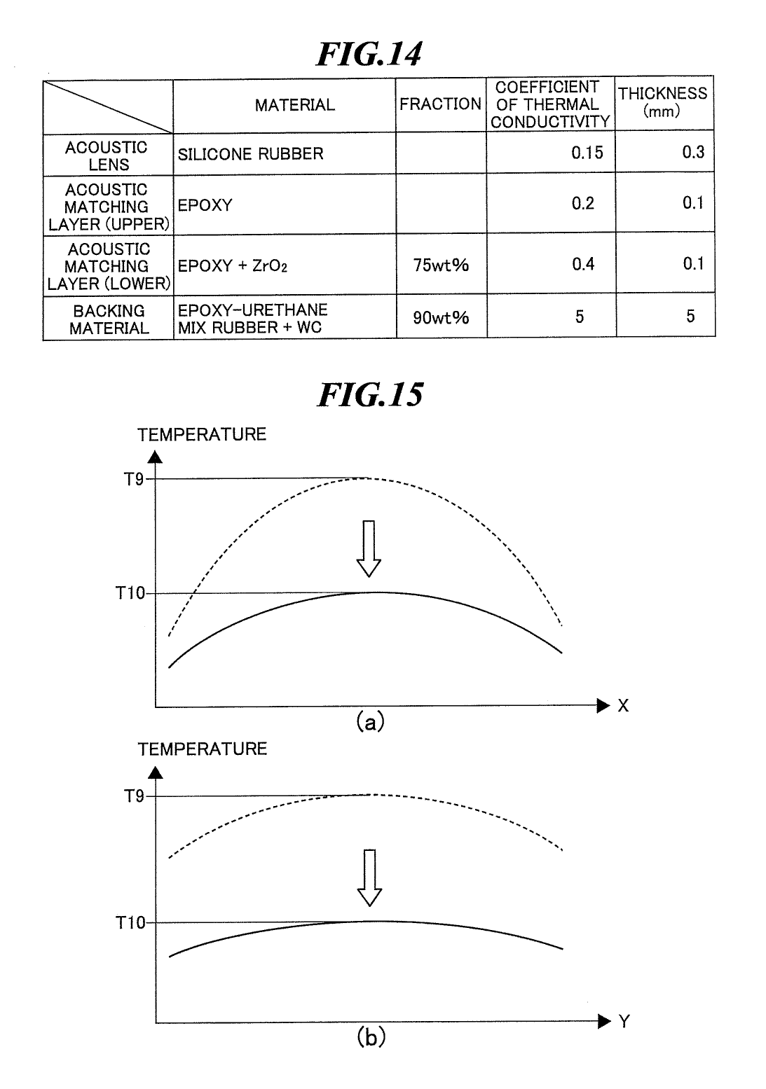

FIG. 14 shows materials and so on of the respective parts in the fifth embodiment of the present invention. As the backing material 1 shown in FIGS. 1 and 2, a material formed by mixing 90 wt % in weight fraction of tungsten carbide (WC) in epoxy-urethane mix rubber is used. The backing material 1 has a coefficient of thermal conductivity of about 5 W/(m·K), and a thickness of 5 mm. As the acoustic matching layer (the lower layer) 4a, a material formed by mixing 75 wt % in weight fraction of zirconia (ZrO₂) in epoxy resin is used. The acoustic matching layer (the lower layer) 4a has a coefficient of thermal conductivity of about 0.4 W/(m·K), and a thickness of 0.1 mm. As the acoustic matching layer (the upper layer) 4b, epoxy resin is used. The acoustic matching layer (the upper layer) 4b has a coefficient of thermal conductivity of about 0.2 W/(m·K), and a thickness of 0.1 mm. As the acoustic lens 5, a material of silicone rubber is used. The acoustic lens 5 has a coefficient of thermal conductivity of about 0.15 W/(m·K), and a thickness of 0.3 mm. Thereby, the thermal resistances of the acoustic matching layers 4a and 4b and the acoustic lens 5 provided on the front side of the vibrator array are larger than the thermal resistance of the backing material 1 provided on the back side of the vibrator array.

FIG. 15 shows measurement results of surface temperature of the ultrasonic probe according to the fifth embodiment of the present invention in comparison with those in a conventional case. The measurement was made by measuring the surface temperature of the acoustic lens in the air at a temperature of 23° C. FIG. 15 (a) shows a temperature distribution in the X-axis direction, which passes through the point of peak temperature on the surface of the acoustic lens, and FIG. 15 (b) shows a temperature distribution in the Y-axis direction, which passes through the point of peak temperature on the surface of the acoustic lens.

In FIGS. 15 (a) and 15 (b), the broken lines show measurement results of surface temperature of the conventional ultrasonic probe, and solid lines show measurement results of surface temperature of the ultrasonic probe according to the fifth embodiment. While the peak temperature T9 in the conventional ultrasonic probe was 39° C., the peak temperature T10 in the ultrasonic probe according to the fifth embodiment was greatly reduced to 28° C.

Next, a modified example of the fifth embodiment of the present invention will be explained. In the modified example, the piezoelectric vibrator has a multilayered structure as shown in FIG. 5, and the rest of the configuration is the same as that in the fifth embodiment.

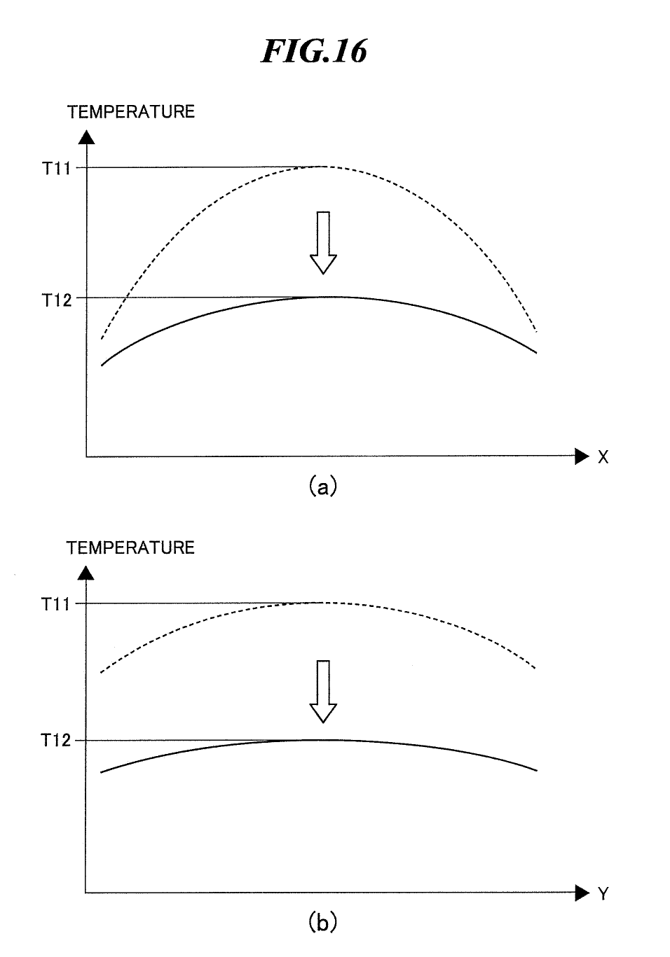

FIG. 16 shows measurement results of surface temperature of the ultrasonic probe according to the modified example of the fifth embodiment of the present invention in comparison with those in a conventional case. The measurement was made by measuring the surface temperature of the acoustic lens in the air at a temperature of 23° C. FIG. 16 (a) shows a temperature distribution in the X-axis direction, which passes through the point of peak temperature on the surface of the acoustic lens, and FIG. 16 (b) shows a temperature distribution in the Y-axis direction, which passes through the point of peak temperature on the surface of the acoustic lens.

In FIGS. 16 (a) and 16 (b), the broken lines show measurement results of surface temperature of the conventional ultrasonic probe, and solid lines show measurement results of surface temperature of the ultrasonic probe according to the modified example of the fifth embodiment. The peak temperature T11 in the conventional ultrasonic probe was 77° C., while the peak temperature T12 in the ultrasonic probe according to the modified example of the fifth embodiment was 38° C. According to the modified example of the fifth embodiment, even when the amounts of heat generated in multilayered piezoelectric vibrators become larger, the heat generated in the piezoelectric vibrators can be rapidly released to the backing material by providing the anisotropic heat conducting material between the piezoelectric vibrators, and thereby, the peak temperature rise in the vibrator array can be suppressed.

Next, the sixth embodiment of the present invention will be explained. In the sixth embodiment, plural piezoelectric vibrators arranged in the X-axis direction and the Y-axis direction form a two-dimensional vibrator array

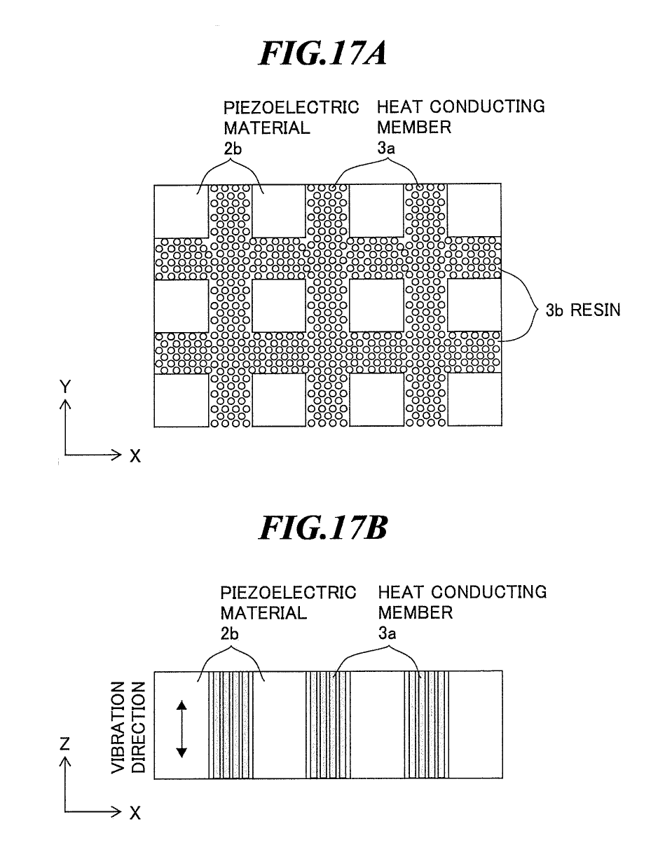

FIG. 17A is a plan view of a composite piezoelectric material in an ultrasonic probe according to the sixth embodiment of the present invention, and FIG. 17B is a side view of the composite piezoelectric material in the ultrasonic probe according to the sixth embodiment of the present invention. Here, a length of each side of a piezoelectric material 2b (in the X-axis direction and the Y-axis direction) is 250 μm, and the thickness of the piezoelectric material 2b (in the Z-axis direction) is 600 μm. The polarization direction of the piezoelectric material 2b is the Z-axis direction. The rest of the configuration is the same as that of the fifth embodiment.

An anisotropic heat conducting material provided between the plural piezoelectric materials 2b forming the vibrator array includes plural heat conducting members 3a arranged substantially in parallel to the vibration direction of the piezoelectric vibrator (the Z-axis direction), and resins 3b filling between the heat conducting members 3a. The heat conducting member 3a has a fibrous or rod-like shape for higher heat conductivity in one direction.

As below, the case where a carbon fiber is used as the heat conducting member 3a and an epoxy resin is used as the resin 3b will be explained. The diameter of each carbon fiber is about 10 μm. The resins 3b are formed by pouring the epoxy resin into spaces between the plural fibers and curing it. The volume fraction of carbon fibers in the anisotropic heat conducting material 3 is preferably from 20% to 78%, and 40% in the embodiment.

The coefficient of thermal conductivity of carbon fiber is about 800 W/(m·K), and the coefficient of thermal conductivity of epoxy resin is about 0.2 W/(m·K). Therefore, the coefficient of thermal conductivity of the anisotropic heat conducting material 3 is about 320 W/(m·K) with respect to the longitudinal direction of the carbon fiber and about 0.33 W/(m·K) with respect to the direction perpendicular to the longitudinal direction of the carbon fiber, and they are greatly improved compared to about 0.2 W/(m·K) in the conventional case of the epoxy resin only. In the sixth embodiment, the coefficient of thermal conductivity in the Z-axis direction is remarkably improved.

Next, a modified example of the sixth embodiment of the present invention will be explained. In the modified example, the piezoelectric vibrator has a multilayered structure as is in the case shown in FIG. 5, and the rest of the configuration is the same as that in the sixth embodiment.

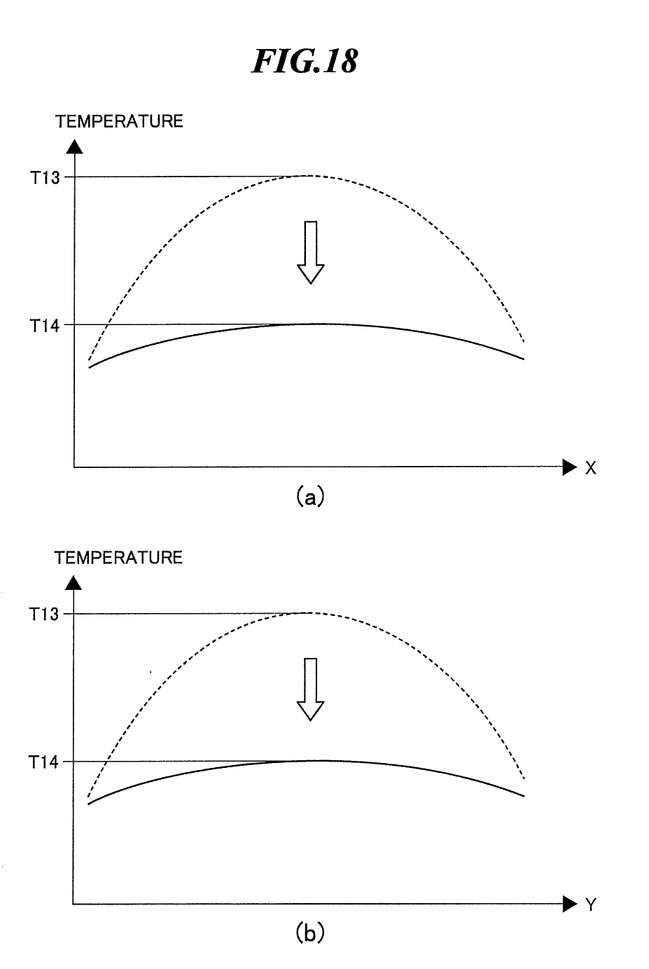

FIG. 18 shows measurement results of surface temperature of the ultrasonic probe according to the modified example of the sixth embodiment of the present invention in comparison with those in a conventional case. The measurement was made by measuring the surface temperature of the acoustic lens in the air at a temperature of 23° C. FIG. 18 (a) shows a temperature distribution in the X-axis direction, which passes through the point of peak temperature on the surface of the acoustic lens, and FIG. 18 (b) shows a temperature distribution in the Y-axis direction, which passes through the point of peak temperature on the surface of the acoustic lens.

In FIGS. 18 (a) and 18 (b), the broken lines show measurement results of surface temperature of the conventional ultrasonic probe, and solid lines show measurement results of surface temperature of the ultrasonic probe according to the modified example of the sixth embodiment. The peak temperature T13 in the conventional ultrasonic probe was 70° C., while the peak temperature T14 in the ultrasonic probe according to the modified example of the sixth embodiment was 33° C. According to the modified example of the sixth embodiment, even when the amounts of heat generated in multilayered piezoelectric vibrators become larger, the heat generated in the piezoelectric vibrators can be rapidly released to the backing material by providing the anisotropic heat conducting material between the piezoelectric vibrators, and thereby, the peak temperature rise in the vibrator array can be suppressed.

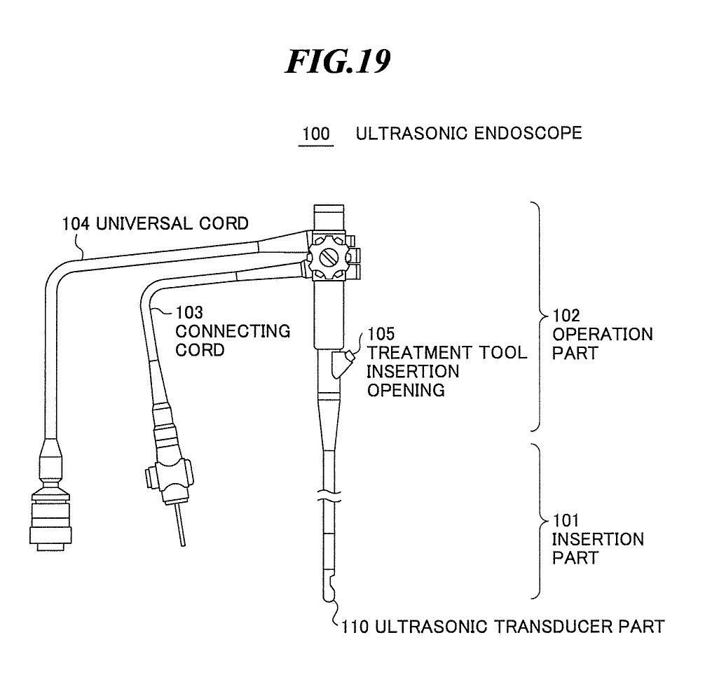

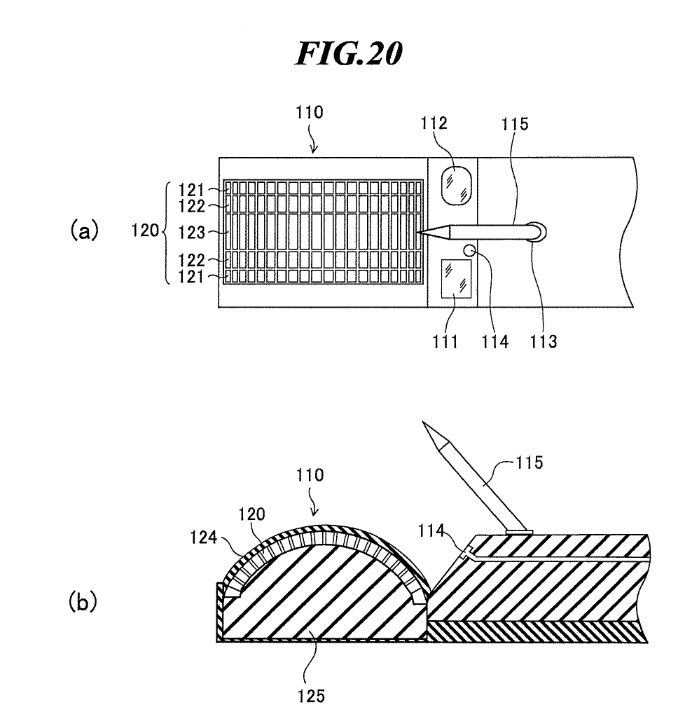

Next, an ultrasonic endoscope according to one embodiment of the present invention will be explained with reference to FIGS. 19 and 20. The ultrasonic endoscope refers to an apparatus provided with an ultrasonic transducer part at the leading end of an insertion part of an endoscopic examination unit for optically observing the interior of the body cavity of the object.

FIG. 19 is a schematic diagram showing an appearance of the ultrasonic endoscope according to the one embodiment of the present invention. As shown in FIG. 19, an ultrasonic endoscope 100 includes an insertion part 101, an operation part 102, a connecting cord 103, and a universal cord 104. The insertion part 101 of the ultrasonic endoscope 100 is an elongated tube formed of a material having flexibility for insertion into the body of the object. An ultrasonic transducer part 110 is provided at the leading end of the insertion part 101. The operation part 102 is provided at the base end of the insertion part 101, connected to the ultrasonic diagnostic apparatus main body via the connecting cord 103, and connected to a light source unit via the universal cord 104. A treatment tool insertion opening 105 for inserting a treatment tool or the like into the insertion part 101 is provided in the operation part 102.

FIG. 20 is an enlarged schematic diagram showing the leading end of the insertion part shown in FIG. 19. FIG. 20 (a) is a plan view showing the upper surface of the leading end of the insertion part 101, and FIG. 20 (b) is a side sectional view showing the side surface of the leading end of the insertion part 101. In FIG. 20 (a), an acoustic matching layer 124 shown in FIG. 20 (b) is omitted.

As shown in FIG. 20, at the leading end of the insertion part, the ultrasonic transducer part 110, an observation window 111, an illumination window 112, a treatment tool passage opening 113, and a nozzle hole 114 are provided. A punctuation needle 115 is provided in the treatment tool passage opening 113. In FIG. 20 (a), an objective lens is fit in the observation window 111, and an input end of an image guide or a solid-state image sensor such as a CCD camera is provided in the imaging position of the objective lens. These configure observation optics. Further, an illumination lens for outputting illumination light to be supplied from the light source unit via a light guide is fit in the illumination window 112. These configure illumination optics.

The treatment tool passage opening 113 is a hole for leading out a treatment tool or the like inserted from the treatment tool insertion opening 105 provided in the operation part 102 shown in FIG. 19. Various treatments are performed within a body cavity of the object by projecting the treatment tool such as the punctuation needle 115 or forceps from the hole and operating it with the operation part 102. The nozzle hole 114 is provided for injecting a liquid (water or the like) for cleaning the observation window 111 and the illumination window 112.

The ultrasonic transducer part 110 includes a convex-type multi row vibrator array 120, and the vibrator array 120 has plural ultrasonic transducers (piezoelectric vibrators) 121-123 arranged in five rows on a curved surface. As shown in FIG. 20 (b), the acoustic matching layer 124 is provided on the front side of the vibrator array 120. An acoustic lens is provided on the acoustic matching layer 124 according to need. Further, a backing material 125 is provided on the back side of the vibrator array 120.

In FIG. 20, the convex-type multi row array is shown as the vibrator array 120, however, a radial-type ultrasonic transducer part in which plural ultrasonic transducers are arranged on a cylindrical surface or an ultrasonic transducer part in which plural ultrasonic transducers are arranged on a spherical surface may be used. In the embodiment, a composite piezoelectric material including an anisotropic heat conducting material provided between plural piezoelectric materials forming the vibrator array 120 is used as in the ultrasonic probe according to the first embodiment to the modified example of sixth embodiment of the present invention.

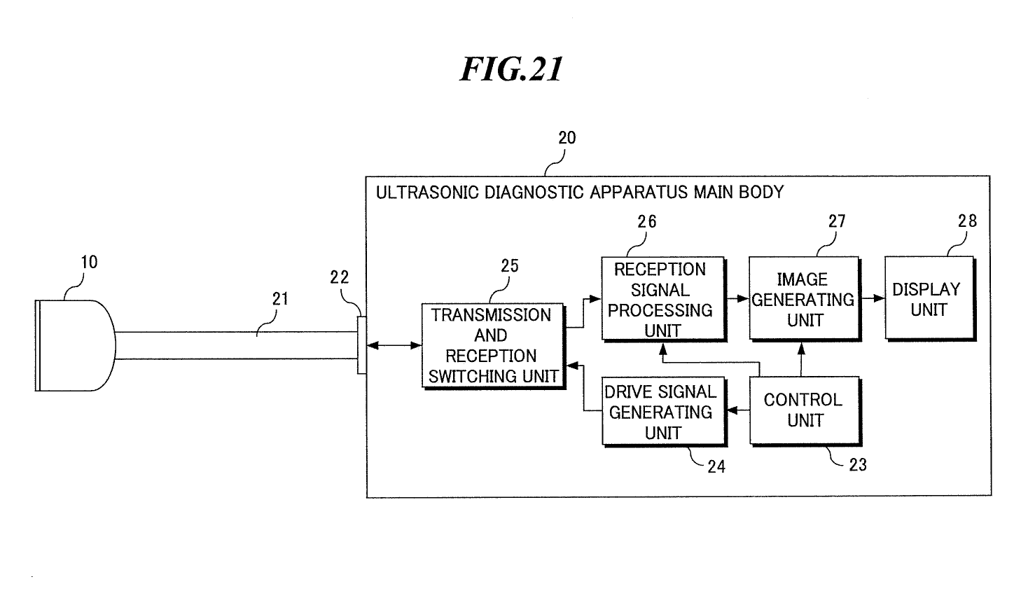

FIG. 21 shows an ultrasonic diagnostic apparatus including the ultrasonic probe or the ultrasonic endoscope according to the respective embodiments of the present invention and an ultrasonic diagnostic apparatus main body. Here, an ultrasonic diagnostic apparatus using the ultrasonic probe will be explained as an example.

As shown in FIG. 21, the ultrasonic probe 10 is electrically connected to the ultrasonic endoscopic apparatus main body 20 via an electric cable 21 and an electric connector 22. The electric cable 21 transmits drive signals generated in the ultrasonic diagnostic apparatus main body 20 to the respective ultrasonic transducers and transmits reception signals outputted from the respective ultrasonic transducers to the ultrasonic diagnostic apparatus main body 20.

The ultrasonic diagnostic apparatus main body 20 includes a control unit 23 for controlling the operation of the entire ultrasonic diagnostic apparatus, a drive signal generating unit 24, a transmission and reception switching unit 25, a reception signal processing unit 26, an image generating unit 27, and a display unit 28. The drive signal generating unit 24 includes plural drive circuits (pulsers or the like), for example, and generates drive signals to be used for respectively driving the plural ultrasonic transducers. The transmission and reception switching unit 25 switches output of drive signals to the ultrasonic probe 10 and input of reception signals from the ultrasonic probe 10.

The reception signal processing unit 26 includes plural preamplifiers, plural A/D converters, and a digital signal processing circuit or CPU, for example, and performs predetermined signal processing of amplification, phase matching and addition, detection, or the like on the reception signals outputted from the respective ultrasonic transducers. The image generating unit 27 generates image data representing an ultrasonic image based on the reception signals on which the predetermined signal processing has been performed. The display unit 28 displays the ultrasonic image based on thus generated image data.

1. A composite piezoelectric material comprising:
plural piezoelectric materials arranged along one of a flat surface and a curved surface; and
an anisotropic heat conducting material having a higher coefficient of thermal conductivity in at least one direction and provided between said plural piezoelectric materials and/or at outer peripheries of said plural piezoelectric materials.

2. The composite piezoelectric material according to claim 1, wherein said anisotropic heat conducting material is provided between said plural piezoelectric materials and at the outer peripheries of said plural piezoelectric materials.

3. The composite piezoelectric material according to claim 1, wherein said anisotropic heat conducting material includes a fibrous material and a resin.

4. The composite piezoelectric material according to claim 3, wherein a coefficient of thermal conductivity of said fibrous material in a longitudinal direction is higher than a coefficient of thermal conductivity of said resin.

5. The composite piezoelectric material according to claim 3, wherein said fibrous material is oriented such that the longitudinal direction of said fibrous material and said one of the flat surface and the curved surface are substantially in parallel.

6. The composite piezoelectric material according to claim 3, wherein said fibrous material is oriented such that the longitudinal direction of said fibrous material and said one of the flat surface and the curved surface are substantially perpendicular.

7. The composite piezoelectric material according to claim 3, wherein said fibrous material contains selected one of carbon fiber, carbon nanotube, gold (Au), silver (Ag), copper (Cu), aluminum (Al), silicon carbide (SiC), aluminum nitride (AlN), tungsten carbide (WC), boron nitride (BN), and alumina (Al₂O₃).

8. The composite piezoelectric material according to claim 1, wherein each of said plural piezoelectric materials includes plural piezoelectric material layers alternately stacked with at least one internal electrode layer in between.

9. An ultrasonic probe to be used for transmitting or receiving ultrasonic waves, said ultrasonic probe comprising:
a vibrator array including a composite piezoelectric material having plural piezoelectric materials arranged along one of a flat surface and a curved surface, and an anisotropic heat conducting material having a higher coefficient of thermal conductivity in at least one direction and provided between said plural piezoelectric materials and/or at outer peripheries of said plural piezoelectric materials;
an acoustic matching layer and/or an acoustic lens provided on a first surface of said vibrator array; and
a backing material provided on a second surface opposite to the first surface of said vibrator array.

10. The ultrasonic probe according to claim 9, wherein a coefficient of thermal conductivity of said backing material is not less than 10 times a coefficient of thermal conductivity of one of said acoustic matching layer and said acoustic lens.

11. The ultrasonic probe according to claim 9, wherein thermal resistances of said acoustic matching layer and said acoustic lens provided on the first surface of said vibrator array are larger than a thermal resistance of said backing material provided on the second surface of said vibrator array.

12. An ultrasonic endoscope including an insertion part formed of a material having flexibility to be used by being inserted into a body cavity of an object to be inspected, said ultrasonic endoscope comprising in said insertion part:
a vibrator array including a composite piezoelectric material having plural piezoelectric materials arranged along one of a flat surface and a curved surface, and an anisotropic heat conducting material having a higher coefficient of thermal conductivity in at least one direction and provided between said plural piezoelectric materials and/or at outer peripheries of said plural piezoelectric materials;
an acoustic matching layer and/or an acoustic lens provided on a first surface of said vibrator array;
a backing material provided on a second surface opposite to the first surface of said vibrator array;
illuminating means for illuminating an interior of the body cavity of the object; and
imaging means for optically imaging the interior of the body cavity of the object.

13. The ultrasonic endoscope according to claim 12, wherein a coefficient of thermal conductivity of said backing material is not less than 10 times a coefficient of thermal conductivity of one of said acoustic matching layer and said acoustic lens.

14. The ultrasonic endoscope according to claim 12, wherein thermal resistances of said acoustic matching layer and said acoustic lens provided on the first surface of said vibrator array are larger than a thermal resistance of said backing material provided on the second surface of said vibrator array.

15. An ultrasonic diagnostic apparatus comprising:
an ultrasonic probe to be used for transmitting or receiving ultrasonic waves, said ultrasonic probe having a vibrator array including a composite piezoelectric material having plural piezoelectric materials arranged along one of a flat surface and a curved surface, and an anisotropic heat conducting material having a higher coefficient of thermal conductivity in at least one direction and provided between said plural piezoelectric materials and/or at outer peripheries of said plural piezoelectric materials, an acoustic matching layer and/or an acoustic lens provided on a first surface of said vibrator array, and a backing material provided on a second surface opposite to the first surface of said vibrator array;
drive signal supply means for supplying drive signals to said vibrator array; and
signal processing means for generating image data representing an ultrasonic image by processing reception signals outputted from said vibrator array.

16. An ultrasonic diagnostic apparatus comprising:
an ultrasonic endoscope including an insertion part formed of a material having flexibility to be used by being inserted into a body cavity of an object to be inspected, said ultrasonic endoscope having in said insertion part a vibrator array including a composite piezoelectric material having plural piezoelectric materials arranged along one of a flat surface and a curved surface, and an anisotropic heat conducting material having a higher coefficient of thermal conductivity in at least one direction and provided between said plural piezoelectric materials and/or at outer peripheries of said plural piezoelectric materials, an acoustic matching layer and/or an acoustic lens provided on a first surface of said vibrator array, a backing material provided on a second surface opposite to the first surface of said vibrator array, illuminating means for illuminating an interior of the body cavity of the object, and imaging means for optically imaging the interior of the body cavity of the object;
drive signal supply means for supplying drive signals to said vibrator array; and
signal processing means for generating image data representing an ultrasonic image by processing reception signals outputted from said vibrator array.
